(19) United States
(12) Patent Application Publication
(10) Pub. No.: US 2023/0104641 A1
(43) Pub. Date: Apr. 6, 2023

(54) Real-time Patient Monitoring for Live Intervention Adaptation

(71) Applicant: Koa Health B.V., Barcelona (ES)

(72) Inventors: Albert Garcia i Tormo, Barcelona (ES); Nicola Hemmings, Bristol (GB); Teodora Sandra Buda, Barcelona (ES); Roger Garriga Calleja, Barcelona (ES); Federico Lucchesi, Rome (IT); Giovanni Maffei, Barcelona (ES)

(21) Appl. No.: 17/494,785

(22) Filed: Oct. 5, 2021

(51) Int. Cl. G16H 20/70; A61B 5/024; A61B 5/01; A61B 5/08; A61B 5/0531; A61B 5/11; A61B 5/16; A61B 5/00; G16H 10/20

(52) U.S. Cl. CPC G16H 20/70; A61B 5/024; A61B 5/01; A61B 5/0816; A61B 5/0531; A61B 5/1118; A61B 5/162; A61B 5/163; A61B 5/4809; A61B 5/165; G16H 10/20

(57) ABSTRACT

A system for monitoring the reaction of a user and for adjusting output content based on the user's reaction includes an output unit, a monitoring unit, a synchronization unit, an analysis unit and a control unit. The output unit presents content to the user. The monitoring unit monitors a user parameter during a period during which a first content is presented to the user in order to obtain monitoring data from the user. The monitoring data is synchronized during the period with the first content so as to link in time the monitoring data and the first content. The analysis unit analyzes the monitoring data and links it to the first content in order to determine the user's reaction to the first content. The control unit controls the output unit to present a second content to the user that is selected based on the user's reaction to the first content.

### TECHNICAL FIELD

The present invention relates to a system and method for monitoring the reaction of a user to a given content and adjusting output content accordingly, preferably in real-time.

### BACKGROUND

As the recent COVID-19 pandemic has doubled the rates of common mental health disorders such as depression and anxiety, there is a large and growing unmet need to remedy undesired symptoms of mental health conditions in the population. It is estimated that around 1 in 5 (21%) adults experienced some form of depression in early 2021. This is an increase compared to a comparable period up to November 2020 (19%) and more than double that observed before the COVID-19 pandemic (10%).

This increase in adverse mental health conditions has put a strain on mental health care professionals such as therapists and psychologists, whose numbers have remained constant. In addition, contact restrictions due to the pandemic have often exacerbated mental health disorders and have also posed a hurdle to treatment, as patients could not easily meet mental health care professionals in person.

In addition, conventional, standardized questionnaires, such as the WHO-5 well-being index, are the basis for the assessment of the mental state of a user. However, despite being the standard, the input gathered by such questionnaires is subjective and prone to biases and even misuse.

It is an object of the present invention to alleviate or completely eliminate the drawbacks associated with existing methods of delivering mental health therapies and treatments. In particular, it is an object of the present invention to ensure that all people receive adequate assistance with their mental health conditions without putting an undue strain on mental health care professionals.

### SUMMARY

A system or method according to the present disclosure enables the mental health state of an individual user to be accurately determined and the individual user to receive tailored mental health care recommendations and resources, such as customized content that is displayed during desensitization treatment for anxiety disorders. The customized content is presented to the user wherever the user is in the user's clinical trajectory and is presented as early as possible in that journey.

The present disclosure relates to a system for monitoring a reaction of a user and adjusting output content accordingly. The system includes an output unit, a monitoring unit, a synchronization unit, an analysis unit and a control unit. The output unit is configured to present content to the user. The monitoring unit is configured to monitor a parameter of the user during a time period in which first content is presented to the user via the output unit in order to obtain monitoring data from the user. The synchronization unit is configured to synchronize the monitoring data obtained by the monitoring unit during the time period in which the first content is presented by the output unit to thereby link in time the monitoring data and the first content. The analysis unit is configured to analyze the monitoring data obtained by the monitoring unit and to link the data to the first content to determine the reaction of the user to the first content. The control unit is configured to control the output unit to present a second content to the user. The second content is selected based on the determined reaction of the user to the first content.

A system for monitoring the reaction of a user and for adjusting output content based on the user's reaction includes an output unit, a monitoring unit, a synchronization unit, an analysis unit and a control unit. The output unit presents content to the user. The monitoring unit monitors a parameter of the user during a time period during which a first content is presented to the user via the output unit in order to obtain monitoring data from the user. The synchronization unit synchronizes the monitoring data obtained by the monitoring unit during the time period with the first content that is presented by the output unit so as to link in time the monitoring data and the first content. The analysis unit analyzes the monitoring data obtained by the monitoring unit and links the monitoring data to the first content presented to the user in order to determine the reaction of the user to the first content. The control unit controls the output unit to present a second content to the user that is selected based on the reaction of the user to the first content.

A method for monitoring a reaction of a user and for adjusting output content accordingly involves presenting first and second content to the user. The first content is presented to the user using an output unit. A parameter of the user is monitored during a time period during which the first content is presented to the user using the output unit in order to obtain monitoring data from the user. The parameter is a physiological parameter, a behavioral parameter, or a parameter indicative of a conscious state of the user. The monitoring data regarding the parameter that is obtained by the monitoring unit during the time period is synchronized such that the first content that is presented by the output unit is linked in time to the monitoring data. The monitoring data obtained by the monitoring unit is analyzed and linked to the first content to determine a reaction of the user to the first content using an analysis unit. A control unit controls the output unit to present a second content to the user, which is selected by the control unit based on the reaction of the user to the first content.

Other embodiments and advantages are described in the detailed description below. This summary does not purport to define the invention. The invention is defined by the claims.

### BRIEF DESCRIPTION OF THE DRAWINGS

The accompanying drawings, where like numerals indicate like components, illustrate embodiments of the invention.

FIG. 1 is a schematic diagram of a user reaction monitoring system that is part of a computing system that implements a smartphone app.

FIG. 2 shows an exemplary embodiment of the system according to the present disclosure.

FIG. 3 shows an example of an iterative analysis by an analysis unit of monitoring data obtained by a monitoring unit.

FIG. 4 shows an exemplary embodiment of method according to the present disclosure including exemplary physiological data.

FIG. 5 shows an exemplary sequence of contents to be presented to the user based on the determined reaction of the user.

### DETAILED DESCRIPTION

Reference will now be made in detail to some embodiments of the invention, examples of which are illustrated in the accompanying drawings.

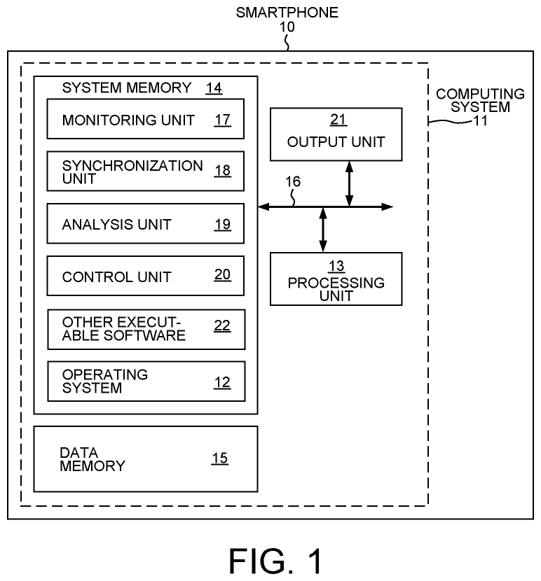

FIG. 1 is a schematic diagram of the components of an application program running on a smartphone 10, which is a mobile telecommunications device. The mobile application (app) forms part of a computing system 11. In one embodiment, the mobile app runs as modules or units of an application program on the computing system 11. In another embodiment, at least some of the functionality of the mobile app is implemented as part of the operating system 12 of smartphone 10. For example, the functionality can be integrated into the iOS mobile operating system or the Android mobile operating system. In yet another embodiment, at least some of the functionality is implemented on the computing system of a remote server that is accessed over the air interface from smartphone 10. The wireless communication modules of smartphone 10 have been omitted from this description for brevity.

Components of the computing system 11 include, but are not limited to, a processing unit 13, a system memory 14, a data memory 15, and a system bus 16 that couples the various system components including the system memory 14 to the processing unit 13. Computing system 11 also includes machine-readable media used for storing computer readable instructions, data structures, other executable software and other data. Thus, portions of the computing system 11 are implemented as software executing as the mobile app. The mobile app executing on the computing system 11 implements a real-time user reaction monitoring system for presenting content to the user and monitoring the user's reaction to the content.

The real-time user reaction monitoring system comprises various units of the computing system 11, including a monitoring unit 17, a synchronization unit 18, an analysis unit 19, a control unit 20, and an output unit 21. The units of the monitoring system are computer readable instructions and data structures that are stored together with other executable software 22 in system memory 14 of the computing system 11.

The novel monitoring system monitors the reaction of a user in real-time to a given content and then adjusts the output content accordingly. The output unit 21 is configured to present content to the user. The monitoring unit 17 is configured to monitor a parameter of the user during a time period during which a first content is presented to the user via the output unit 21 in order to obtain monitoring data from the user. The synchronization unit 18 is configured to synchronize the monitoring data, which is obtained by the monitoring unit 17 during the time period during which the first content is presented by the output unit 21, with the first content presented by the output unit 21 to thereby link in time the monitoring data and the first content. The analysis unit 19 is configured to analyze the monitoring data obtained by the monitoring unit 17 and to link the monitoring data to the first content presented in order to determine the user's reaction to the first content. The control unit 20 is configured to control the output unit 21 in order to present a second content to the user. The second content is selected based on the determined reaction of the user to the first content.

As an example, the user reaction monitoring system may be used to expose a user to a series of images that are expected to elicit a certain reaction in the user, such as fear of the user when presented with an image of a spider. Depending on the detected reaction of the user to a given image, the next image to be presented to the user will be an image expected to elicit a stronger response of the user. In this example, a moderate reaction (moderately increased heart rate) is detected to a realistic image of a spider, and then the next image depicts a realistic spider sitting on a human hand and is expected to elicit a strongly increased heart rate in the user.

The present disclosure, however, is not limited to a case in which a defined series of content to be presented is used. Instead, the control unit 20 can also be configured to select the next content based on a reaction of the user to a previous content. The series of content presented is not defined in advance, but instead is determined instantaneously. For example, the first content is selected from a first pool of contents, and the second content is selected from a second pool of contents based on a detected reaction of the user to the first content, such as through the monitoring of physiological data or based on input the user actively provides. There is no predefined order of contents to be presented, but rather the series evolves gradually based on the reactions of the user to content previously presented.

The monitoring unit 17 is configured to monitor one or more of the following parameters: a physiological parameter of the user (such as heart rate, respiration rate, pupil dilation, body temperature, skin conductivity), a behavioral parameter (such as an activity profile, sleep pattern, a reaction time, gaze direction, data regarding social interactions), and a parameter data indicative of a conscious state of the user (such as data stemming from questionnaires or data input by the user). The monitoring unit 17 can include one or more sensors configured to measure the parameters to be monitored. The data acquired by the monitoring unit 17 when monitoring the one or more parameters are referred to as monitoring data.

The real-time user reaction monitoring system may be realized in smartphone 10. Smartphones include multiple sensors that can also be used for monitoring the user, for example while the user is using the phone. The sensors provide automatic and unobtrusive measurements of physiological parameters of the user. In particular, the camera of a user's smartphone can be used to monitor different physiological parameters, as this camera provides a close-up of the patient's face while the patient is using the phone. These parameters include, but are not limited to, the instantaneous heart rate (from a photoplethysmogram signal or camera-based measurement) and the instantaneous respiration rate (movement around the chest area, camera-based measurement). The main advantage of using a camera to monitor physiological parameters is that the monitoring is completely unobtrusive and automatic, thereby allowing users to be monitored without the monitoring influencing them (unconditioned measurements) and without requiring them explicitly to provide input.

The monitoring unit 17 monitors a parameter of the user automatically and unobtrusively without the user being required to actively provide input, offers the possibility of monitoring users who cannot easily fill in text-based questionnaires, such as children or people with reading difficulties, and extending the text-based questionnaires with non-textual questions. For instance, images or videos can be presented to the user, and the user's reaction to the images or videos can be monitored. Data indicative of the user's reaction is synchronized or linked in time with the presented content that elicited the reaction. The monitoring unit 17 is configured to monitor the parameter of the user for any desired time period, such as continuously 24 hours per day, or only during the time period during which a first content is displayed, such as for a given number of hours each day.

In one embodiment, in response to the first content being presented to the user, the analysis unit 19 receives data indicative of a conscious state of the user (such as data obtained from questionnaires or data input by the user) and data indicative of a subconscious state of the user (such as physiological data) and compares the data indicative of the conscious state of the user with the data indicative of the subconscious state of the user in order to determine the user's reaction to the first content.

For example, the user may consciously report an absence of fear, but the physiological data may indicate signs of fear, such as an increased heart rate. Considering both data indicative of the conscious state of the user, such as data obtained from questionnaires or data input by the user, and data indicative of a subconscious state of the user, such as physiological data or behavioral data, enables the user's reaction to be more accurately detected.

In one embodiment, the analysis unit 19 is configured to detect changes in a parameter monitored by the monitoring unit 17 relative to that parameter as previously monitored and to determine the user's reaction to the first content based on the detected changes. The analysis unit 19 is configured to detect the absolute value of the parameter monitored by the monitoring unit 17 and to determine the user's reaction to the first content based on the detected absolute value.

The control unit 20 is configured to select the second content during the time period during which the first content is displayed to the user. The control unit 20 is also configured to control the output unit 21 to present the second content immediately after the first content is presented. In other words, the control unit 20 is configured to select the second content in real time, such as during the time period in which the first content is displayed. The control unit 20 is configured to control the output unit 21 in real time to present the second content immediately after the first content is presented.

Alternatively, the control unit 20 is configured to select the second content during a time period during which the first content is displayed. The control unit 20 is configured to control the output unit 21 to present the second content in the future, for example the next time the user interacts with the real-time user reaction monitoring system. In this case, the presented content is not immediately adapted based on the detected reaction of the user, but instead data indicative of the detected reaction of the user is stored, and the selected second content is presented at a desired time in the future. For example, the second time a user interacts with the system a different series of first content and second content is presented than the first time the user interacted with the system.

The control unit 20 is configured to select the second content to elicit a desired reaction of the user. For example, the control unit 20 can select a second content expected to elicit a stronger reaction of the user (e.g., a strong increase in heart rate) or a second content expected to elicit a milder reaction of the user (e.g., a mild increase in heart rate). The second content may be selected to induce a desired reaction in the user or to put the user in a desired mental state, for example to induce a desired level of fear or wellbeing.

The control unit 20 is configured to select a second content expected to elicit a stronger physiological reaction of the user than the first content if the determined physiological reaction of the user to the first content falls within a predetermined tolerance range. The control unit 20 is configured to select a second content expected to elicit a milder physiological reaction of the user than that elicited by the first content if the determined physiological reaction of the user to the first content falls outside the predetermined tolerance range. The control unit 20 is configured to select a second content expected to calm the user, such as a guided relaxation program, if the determined physiological reaction of the user to the first content falls outside of a predetermined tolerance range or exceeds a tolerance threshold.

The monitoring unit 17, the output unit 21 and the synchronization unit 18 are present in one single device, such as a smartphone. The single device includes a synchronization device, such as an internal clock, and the synchronization unit 18 is configured to use the signal of the synchronization device to synchronize the monitoring data obtained by the monitoring unit 17 with the first and second content presented by the output unit 21. The synchronization can involve linking in time the monitoring data with the first and second content, such as by providing corresponding monitoring data and data regarding the presented content with a common time stamp.

The real-time user reaction monitoring system also includes a data memory in which a series of contents to be consecutively presented to the user via the output unit 21 is stored. The control unit 20 is configured to control the output unit 21 to consecutively present the content of the series to the user, and if the reaction of the user determined by the analysis unit 19 to a presented content of the series falls outside a predetermined tolerance range, the control unit 20 interrupts or modifies the consecutive presentation of contents. The tolerance range is defined in terms of the monitoring data and may include a maximum value for the heart rate or respiration rate or a minimum value of the sleep time in case behavioral data is monitored.

The analysis unit 19 can be further configured to automatically determine the mental health state of the user based on the user's reaction to the first content. If the user shows a strong reaction to the first content that is expected to elicit only a mild reaction, the analysis unit 19 can determine that the user is in a general state of agitation or stress in which even relatively mild stimuli elicit a strong reaction. Conversely, if the user is in a relaxed and happy state, a content that is expected to elicit a strong reaction may elicit only a mild reaction.

A method for monitoring the reaction of a user and for adjusting output content accordingly involves monitoring the user's reaction to content. The user is presented with a first content via the output unit 21. A parameter of the user is monitored during a time period in which the first content is presented to the user via the output unit 21 in order to obtain monitoring data from the user. The data regarding the parameter obtained by the monitoring unit 17 during a period in which the first content is presented by the output unit 21 is synchronized with the first content presented by the output unit 21 via the synchronization unit 18 to thereby link in time the monitoring data and the first content. The monitoring data obtained by the monitoring unit 17 is analyzed. The monitoring data is linked to the first content in order to determine the user's reaction to the first content using the analysis unit 19. The control unit 20 controls the output unit 21 to present a second content to the user. The second content is selected by the control unit 20 based on the reaction of the user to the first content.

The monitoring step of the method involves monitoring one or more of the following parameters: a physiological parameter of the user (such as heart rate, respiration rate, pupil dilation, body temperature, skin conductivity), a behavioral parameter (such as data regarding an activity profile, sleep pattern, a reaction time, gaze direction, data regarding social interactions), and a parameter indicative of a conscious state of the user (such as data obtained from questionnaires or data input by the user). Other data, such as data obtained from electronic health records may also be acquired via the monitoring unit 17.

The method may also include the steps of receiving data about conscious and subconscious states of the user and comparing the data for those states to determine the user's reaction. Data is received via the analysis unit 19 indicative of a conscious state of the user, such as data obtained from questionnaires or data input by the user. The analysis unit 19 also receives data indicative of a subconscious state of the user, such as physiological data. The data indicative of the conscious state of the user is compared with the data indicative of the subconscious state of the user in order to determine the user's reaction to the first content.

The method may also include the steps of using the analysis unit 19 to detect changes in the parameter monitored by the monitoring unit 17 relative to that parameter measured previously and determining the user's reaction to the first content based on the detected changes. The analysis unit 19 is used to detect an absolute value of the parameter monitored by the monitoring unit 17 and to determine the user's reaction to the first content based on the detected absolute value.

The second content is selected during a time period in which the first content is displayed. The output unit 21 is controlled to present the second content immediately after the first content is presented. The second content is selected in real time, for example during the time period in which the first content is displayed. The output unit 21 is controlled in real time to present the second content immediately after the first content is presented.

The second content is selected during the time period during which the first content is displayed, and the output unit 21 is controlled to present the second content in the future, for example the next time the user interacts with the system. In this case, the content presented is not immediately adapted based on the detected reaction of the user, but instead data indicative of the detected reaction of the user or data indicative of the selected second content is stored, and the selected second content is presented at a desired time in the future. For example, the second time a user interacts with the user's smartphone, a different series of first content and second content is presented than was presented the first time the user interacted with the user's smartphone.

The second content is selected to elicit a desired reaction in the user. For example, the control unit 20 can select a second content expected to elicit a stronger reaction of the user (such as a strong increase in heart rate) or a second content expected to elicit a milder reaction of the user (such as a mild increase in heart rate). In other words, the second content may be selected to induce a desired reaction in the user or to put the user in a desired mental state, for example to induce a desired level of fear or wellbeing.

A second content expected to elicit a stronger physiological reaction of the user than that elicited by the first content is selected if the determined physiological reaction of the user to the first content falls within a predetermined tolerance range. However, a second content expected to elicit a milder physiological reaction of the user than that elicited by the first content is selected if the determined physiological reaction of the user to the first content falls outside of the predetermined tolerance range. A second content expected to calm the user is selected, such as a guided relaxation program, if the determined physiological reaction of the user to the first content falls outside of a predetermined tolerance range or exceeds a predetermined threshold.

The synchronizing step of the method is performed using a signal of a synchronization device, such as an internal clock, of a single device that includes the monitoring unit 17, the output unit 21 and the synchronization unit 18. The synchronizing step synchronizes the monitoring data obtained by the monitoring unit 17 with the first and/or second content presented by the output unit 21. For example, the single device is smartphone 10.

The method includes the step of storing in a memory a predetermined series of contents to be consecutively presented to the user via the output unit 21. The output unit 21 is controlled to consecutively present the contents of the series to the user and, if the reaction of the user determined by the analysis unit 19 to a presented content of the predetermined series falls outside a defined tolerance range, to interrupt or modify the consecutive presentation of the contents.

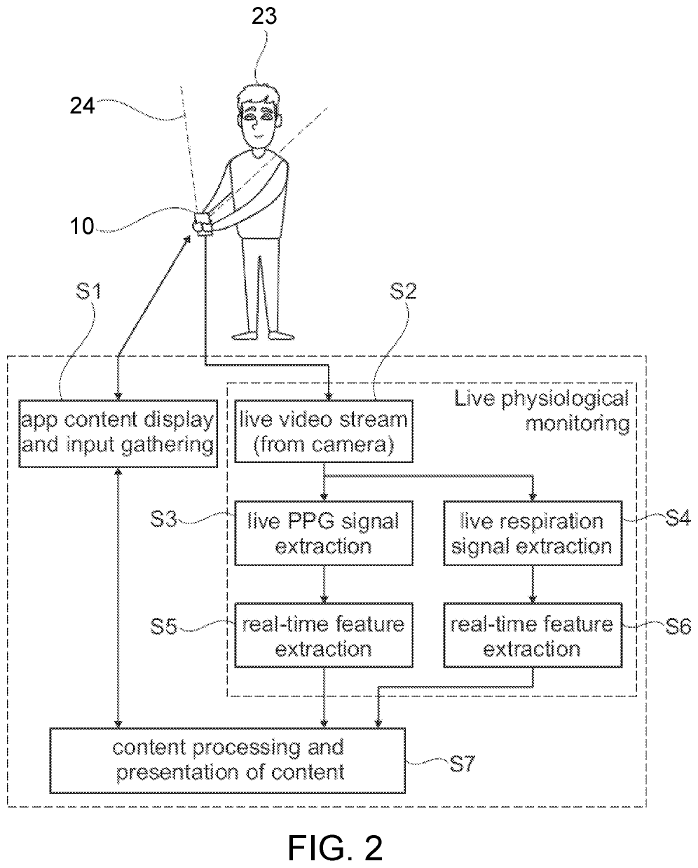

FIG. 2 shows a user 23 interacting with his smartphone 10 by holding the smartphone such that the face and chest of the user are present in an acquisition range 24 of the smartphone, which allows the front camera of the smartphone to be used to monitor parameters, for example physiological parameters, of user 23. In this example, the real-time user reaction monitoring system is realized on the smartphone 10 of user 23. The smartphone 10 is used to perform the method according to the present disclosure.

In step S1, the output unit 21, in this case the screen of the smartphone, presents the user 23 with a content via a mobile application. At the same time, the app prompts the user 23 to rate his current mental state or state of wellbeing and to provide other input indicative of the conscious state of the user.

While the user 23 is interacting with the smartphone 10, in step S2 a live video stream is acquired using the front camera of the smartphone 10 to monitor the user. Preferably, a color video stream is acquired. The acquisition of the video stream in S2 does not require any active input of the user and might not even be noticed by the user.

In step S3, a signal is acquired from the video stream indicating the heart rate of the patient. One example of the signal is a photoplethysmogram (PPG). In general, different vital signs can be monitored with regular smartphone cameras, including pulse and respiration, as well as activity, sleep and other aspects related to the user's health. A photoplethysmogram (PPG) is a measurement of blood volume changes in the microvascular bed of tissue. A PPG measures changes of color in the skin caused by the pulsatile flow of blood flowing through the body. With each heart beat, the heart pumps a pulse of blood through the arteries; the blood pulse travels through them to the capillaries and, from there, it returns to the heart via the veins. Because the skin has many capillaries (i.e., it is highly perfused), it is feasible optically to measure the pulsatility of the blood flow. Whenever a blood pulse reaches the capillaries, the local increase in the blood flow causes a local increase of light absorption which, in turn, causes a minute color change in the skin. Even though this color change is imperceptible to the naked eye, it can be captured with a digital camera in the form of a PPG signal.

The PPG signal consists of a large constant (DC) component, which corresponds to the main skin absorption, and a pulsatile (AC) low-amplitude component, which corresponds to the variation in blood volume. Typically, the amplitude of the pulsatile component is in the range of 1% compared to the constant component.

Generally, the amplitude of the pulsatile component is very low, even below the resolution in an 8-bit scale, and well below the camera noise level. In order to reject the noise and achieve enough resolution, usually the signal is not measured from just one pixel but averaged over a large number of pixels in a region of interest (RoI). As the PPG signal is strongest at the areas that are most highly perfused, the face and the palms of the hands and the feet are usually the best areas to measure the PPG signal. The raw PPG signal shows variations in light intensity: a burst of blood increases the absorption which results in a decrease of light intensity. The peaks of the raw PPG signal correspond to the moments of minimum blood flow. Typical color cameras capture three different wavelengths: red, green and blue. The light absorption is largest around the green wavelength, which results in a PPG signal of larger amplitude in the green channel than in the blue and the red channels. Thus, preferably, the green channel of the camera is analyzed to measure the user's heart rate. In a healthy subject, all blood pulses pumped by the heart reach all limbs and, in particular, the face and the hand. Consequently, measuring the frequency of the PPG signal at, e.g., a hand, is a way of measuring the heart rate. Furthermore, because the pulse transit time (PTT) does not substantially affect the cycle-to-cycle measurement, it is feasible to measure the Instantaneous Heart Rate (the length of each individual heart cycle, iHR) to evaluate parameters such as the heart rate variability (HRV). Insights about other parameters, such as blood pressure, can also be obtained from the PPG signal.

In step S4, a signal indicating the respiration rate of the patient is also acquired from the video stream. The respiration rate can be extracted from the video stream by monitoring the chest movement of the user.

The signal extraction in steps S3 and S4 occurs live during the acquisition of the video stream and concurrently with the display of a first content on the screen of smartphone 10. Thus, the data obtained in step S2 and analyzed subsequently in steps S3-S6 is synchronized with the first content that is displayed to the user.

In steps S5 and S6, features are extracted from the PPG signal acquired in step S3 and the respiratory signal acquired in step S4, which are the monitoring data from user 23. In each of steps S5 and S6, a defined parameter is extracted that corresponds to a defined numerical that can be used for numerical processing from the video stream acquired in step S2. For example, in step S5, a heart rate in beats per minute (bpm) is extracted from the pulsatile changes in tissue color encoded in the PPG signal. In step S6, for example, a respiration rate in breaths per minute is extracted from the chest movements detected in step S4.

The feature extraction in steps S5 and S6 in this example occurs in real time, i.e., at the same time at which the first content is displayed on smartphone 10.

It is also be possible for the feature extraction and analysis to occur not in real time, but with a time delay relative to the acquisition of the monitoring data. For example, the monitoring data can be stored in data memory 15 for later processing. The monitoring data can then be processed even during a time period during which the user does not interact with his smartphone. The content is then presented at the next time the user 23 interacts with the smartphone 10.

In step S7, the features extracted in steps S5 and S6 are linked in time with the content presented on the smartphone 10 so that the physiological reaction of the user 23 in terms of heart rate and respiration rate can be linked to the first content.

Then in step S7, a second content to be presented to the user is selected based on the detected physiological reaction of the user to the first content. The second content is then presented to the user 23 on the display of the smartphone 10.

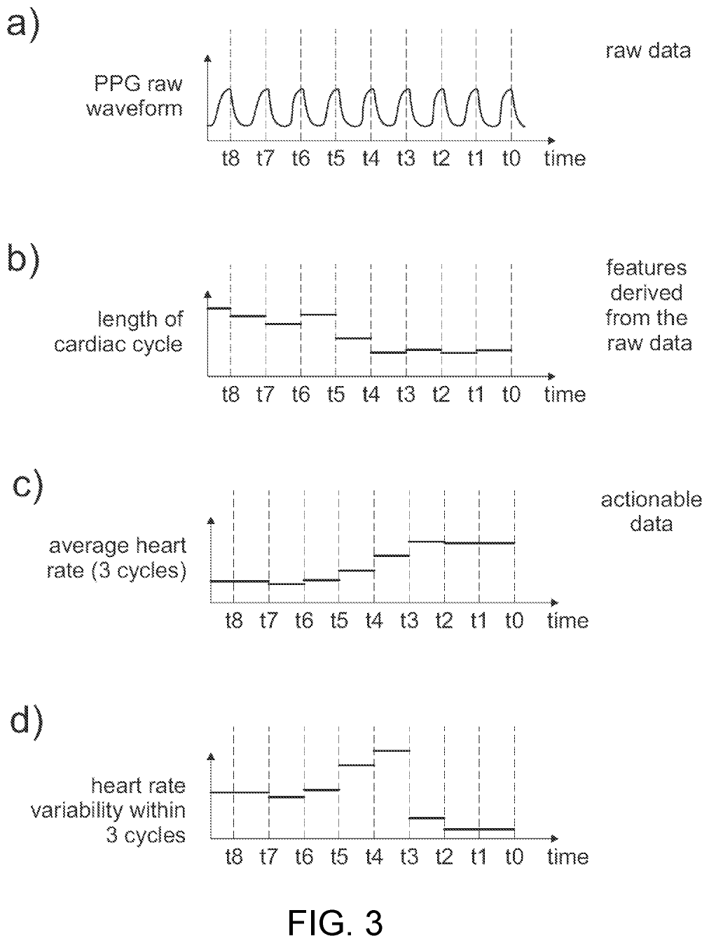

FIG. 3 shows an example of an iterative analysis performed on PPG data acquired by the front camera of the smartphone 10 of the user 23 to extract features useful for subsequent processing (also referred to as actionable data).

Part a) of FIG. 3 shows raw data, in this example a waveform indicating color changes of the user's skin acquired in step S3. In step S5, this raw data is processed to extract the length of the cardiac cycle from the PPG signal as shown in part b) and to obtain actionable data such as the average heart rate of the user in bpm averaged over three cycles as shown in part c) or the average heart rate variability in ms averaged over three cycles as shown in part 2d). For example, the average heart rate shown in part c) of FIG. 3 and the average heart rate variability shown in part d) of FIG. 3 can then be used to determine the user's reaction to the first content. For example, part c) of FIG. 3 shows an increase in average heart rate from t8 to t0, which in this case indicates that the user is experiencing the first content as being agitating or arousing.

A live measurement of the heart rate can be obtained as follows. The face of the user must be located in the acquisition range of the camera. This can easily be achieved using a face detector. Similarly, the face detector can also identify elements in the face, such as the forehead and the cheeks. These three elements (forehead and both cheeks) define the region of interest (RoI) and must be identified in all frames of the video stream acquired by the front camera. The raw PPG signal can be extracted from the live video stream by averaging all the pixels within the RoI per frame. For an improved signal-to-noise ratio, the green, red and blue channels may be independently analyzed and afterwards combined. The result of each frame can be concatenated, thereby creating a time-domain signal (raw PPG signal).

Directly relying the raw PPG signal to determine the user's reaction is not advisable because the information that the raw PPG signal conveys is implicit within a waveform (and thus not actionable) that captures multiple physiological parameters at the same time. The raw PPG signal is thus be split into multiple signals, each of which conveying explicit information of only one physiological feature, such as the heart rate or the heart rate variability (actionable data). A feasible way of obtaining actionable data from the raw PPG signal is to determine the length of each cardiac cycle by locating the peaks in the raw PPG signal, which correspond to the moments of minimum blood flow, and then determining the time distance between peaks to obtain the length of the cardiac cycle. This feature can be further split into an Average Heart Rate (e.g., the inverse of the average length of the last three cardiac cycles, aHR) and the HRV, which is the difference in length between the last two cardiac cycles. These features convey explicit information of only one physiological parameter and therefore are actionable. They can be used to determine the user's reaction to the content displayed on the smartphone 10.

Using a device such as the smartphone 10, the process of feature extraction from raw data can be executed with a very small delay so that the cardiac information is updated and made available for processing within a few milliseconds after each heart beat. This allows any change in the patient's heart beat to be immediately detected by the smartphone 10.

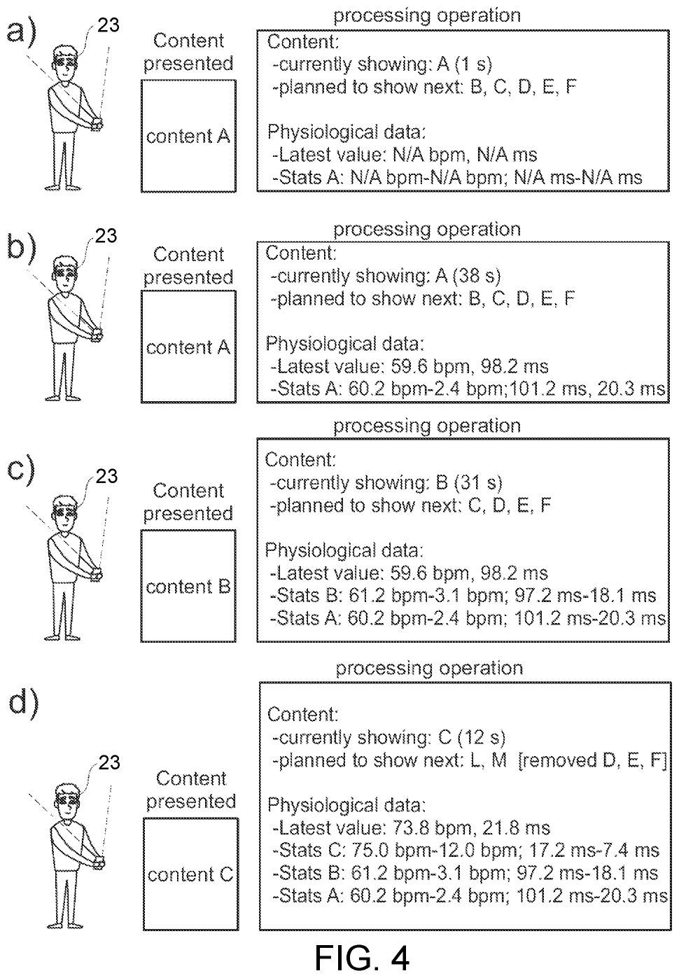

FIG. 4 shows an exemplary embodiment of a system and method for monitoring the user 23 via the front camera of his smartphone 10 and for obtaining PPG data from the monitoring data to extract the average heart rate over three cycles and the heart rate variability over three cycles. The reaction of the user 23 to a content presented to the user is detected based on changes in the extracted heart rate and heart rate variability that occur immediately after the patient is presented with a content. In this example, there is a defined series of content that is consecutively presented to the user, denoted content A-F in FIG. 4.

As shown in part a) of FIG. 4, during a first stage at the beginning of the interaction of the user 23 with his smartphone 10, the user 23 views a content A presented on the smartphone 10. The content A is presented to the user for 1 s. A live video stream of the user is acquired via the front camera of the smartphone 10. As the interaction of the user 23 with the smartphone 10 has only started, there are no data yet available to determine the average heart rate in bpm or the average heart rate variability in ms.

As shown in part b) of FIG. 4, the content A has been presented to the user 23 for 38 s. Data indicating the average heart rate in bpm and the average heart rate variability in ms has been extracted from the live video stream. For example, the most current instantaneous heart rate of the user is 59.6 bpm and the latest heart rate variability is 98.2 ms. In addition, statistical analysis has been performed to obtain the average heart rate, in this example 60.2 bpm, the standard deviation of this average value, in this example 2.4 bpm, the average heart rate variability, in this example 101.2 ms, and the standard deviation of this variability value, in this example 20.3.

The acquired physiological data is associated with the content A that has been presented during the acquisition of the data by pooling. Statistical analysis is then performed on the data in each pool.

Pooling refers to creating a set of pools of data, one for each distinct content being presented in the application. For example, there is a pool of data for content A, a pool of data for content B, and so on. The data acquired in part b) of FIG. is pooled into a pool associated with content A.

Each new physiological datapoint (i.e., each new value of average heart rate (aHR) and heart rate variability (HRV)) is stored in the corresponding pool according to the content presented.

Whenever a new value is added to a pool, two statistical parameters for each of aHR and HRV are evaluated for that pool: the average value and the standard deviation. The statistics are significant when at least a minimum number of data points, e.g., five, has been acquired for that pool.

To compare data, the following criterion can be used: given a datapoint a and a pool with average b and standard deviation c, a behaves similarly to that pool if a∈[b−c, b+c).

Based on this criterion, a criterion for determining the user's reaction to presented content, a potential increase in anxiety can be defined. For example, when the aHR in the current pool is larger than that in the previous pool, and the HRV in the current pool is smaller than that in the previous pool, this indicates that the presentation of the current content induced an increase in heart rate and a decrease in HRV relative to the previous content and thus elicited an increase in anxiety in the user.

The feature extraction and feature analysis may be performed by a machine learning-based system, such as a trained model or neural network, for example a convolutional neural network.

To establish the link between the change in the physiological features and the presented content, it is advantageous to identify the moment in time where these physiological changes occurred and to link them to the content that was presented to the user at that moment. Because both the presentation of the content and the acquisition of the physiological data occur on the same device, a synchronization device such as the internal clock of smartphone 10, can be used to synchronize the various data sources. Each datapoint is accompanied by metadata, such as a timestamp in the data format indicating the time since epoch in seconds. In this way, when comparing datapoints from different inputs (e.g., heart rate and change of presented content), the chronological sequence of the datapoints can be determined simply by comparing the timestamps.

In the example shown in part b) of FIG. 4, the physiological data acquired when the user 23 is being presented with content A indicates that the user is feeling relaxed. The aHR is low (around 60 bpm in average, with less than 5 bpm variation), and the user 23 exhibits respiratory sinus arrhythmia (RSA), a respiration-induced modulation of the instantaneous heart frequency that is common when individuals are relaxed. Due to the RSA, the HRV of the user 23 is large, in the range of 100 ms.

After having displayed content A to the user 23 for a defined time period, as shown in part c) of FIG. 4, content B is displayed to the user 23 on his smartphone 10. When switching to content B, the data pool for content A is closed and the pool for content B is opened so that any new data acquired will be grouped into the pool for content B. After gathering some data, e.g., at least three values of aHR, statistical analysis is performed on the data in the pool for content B. When viewing content B, the average heart rate is 61.2 bpm with a standard deviation of 3.1 bpm, and the average heart rate variability is 97.2 ms with a standard deviation of 18.1 ms.

The statistical values derived from the data in the pool corresponding to contents A and B are compared and are found in this example to be similar or not significantly different. The average value while viewing content B falls into the interval defined by content A +/− the standard deviation (61.2∈[57.8, 62.6). In addition, all instantaneous values are found to be similar.

As shown in part d) of FIG. 4, when the user is presented with content C, the values are no longer similar. It is not necessary to acquire many heart cycles in order to derive statistics because in this case the instantaneous values already indicate a different physiological response. Of course, it is possible to derive statistical values from the datapoints in the pool for content C as well. In this example, as shown in part c) of FIG. 4, the average heart rate of the user viewing content C quickly increases beyond 70 bpm, reaching an instantaneous value of 73.8 bpm and an average value of 75.0 bpm, which is well above the maximum (average plus standard deviation) while the previous contents were being viewed (62.6 bpm and 64.3 bpm respectively).

Likewise, the HRV also shows a significant change in response to content C because the average value of the HRV when the user views content C, 17 ms, is well below the average value minus standard deviation when the user views contents A and B, 80.9 ms and 79.1 ms, respectively. Because of this change in both the aHR and HRV (both values deviating from the values known to be associated with a relaxed state, such as when viewing contents A and B), the reaction of the user 23 to content C can be determined to be an increase in anxiety.

The control unit 20 of the smartphone 10 thus adapts the series of contents to be consecutively presented to the user, for example by removing the initially planned contents D, E and F that are expected to be increasingly provocative to the user, and adds the new contents L and M that are expected to calm the user. The series of content to be presented is thus adapted from A-B-C-D-E-F to A-B-C-L-M based on the detected reaction of the user (increase of anxiety) to content C.

In this example, the criterion to determine an anxiety increase is for both the aHR and the HRV to exceed a certain threshold. Different criteria may be used, which may involve using different physiological data and/or quantifying the measurement into actionable data (see next embodiment). The criteria may be defined so as to detect and identify different reactions of the user.

In addition, instead of providing a binary output (increase in fear: yes or no?), the criterion may provide a multi-level value (i.e., a number) indicating the intensity of detected reaction (e.g., rating of the detected increase in fear on a scale from 1-5). For example, a possible multi-level quantification from only the aHR is first to determine a reference level and then to provide as a quantified output the difference of the current value with respect to the reference level—the higher the value, the higher the anxiety. The reference level can be acquired during the first 30 s, for example, while the user is presented with some relaxing content, even though the reference value need not be acquired every time the user interacts with his smartphone 10.

It is also possible to determine the normal or baseline values for an individual user by analyzing the physiological data over time, such as across multiple days. In this way, an individual starting point or baseline can be defined for an individual user. This baseline does not necessarily need to be based on numbers (e.g., the average heart rate when the patient is feeling relaxed), but can also be based on other features such as the shape of the PPG waveform. Monitoring multiple features at the same time introduces redundancy, which is often advisable in order to reduce the errors in conclusions drawn from the acquired data. Such personalized baseline recordings can also be used to determine which features are most relevant for each particular user, considering that not all features are equally indicative of a given reaction by individual users. For example, for a first user the increase in heart rate is more closely linked to an increase in anxiety, and for a second user the increase in respiration rate is more closely linked to an increase in anxiety.

Any desired parameter of a user may be monitored to obtain data indicative of the user's reaction to a content that is displayed. Still referring to image processing, the amount of head movement or the pupil size are just two parameters. Furthermore, other data may be incorporated as well (synchronized with the content presented), such as accelerometer data, missed taps on the phone or tap intensity.

Another parameter that may provide valuable insights is the reaction time. The reaction time is defined as the time elapsed between a first and a second event. The first event can be the change in the content displayed to the user, and the second event may be a change in a physiological feature. Or the first event may be a change in a physiological feature, and the second event is a user input. Or the first event may be a change in the content displayed in the application, and the second event is a user input.

The user's reaction may also be detectable from the user's voice. Indeed, because vocalization is entirely integrated into both a person's central and autonomic nervous system, there is a link between the voice output and the associated psychological and physiological state of the user. Voice data can be captured using a microphone, processed within milliseconds and then used as disclosed above. Similar to video-based signals, voice or audio data (i.e., an audio signal) can be analyzed by first extracting the relevant features from the audio data that is linked to the target outcome. Then an arousal level of the user, for example, can be derived by analyzing the feature values and, if applicable, the content can be customized. For example, stress can be detected by analyzing the voice of the patient. Examples of features that can be extracted are: respiration rate, articulation rate, word duration, vowel duration, respiration time between words or sentences, voice onset time, hitter, shimmer, signal to noise ratio, harmonic to noise ratio, mean F0 SD, F0 peaks, and F0 floor values. Based on any changes detected in the voice-based extracted features, the level of stress can be quantified, either in a binary or multi-level manner.

In addition, data from wearables or any other sources separate from the smartphone 10 may be used. For example, a chest band or an additional hand-held PPG sensor may be used, and the data is made available in real-time and synchronized with the smartphone 10.

Generally, based on the acquired data, mismatches between the physiological (and thus spontaneous) reaction of a user and the conscious reaction of the user (detected based on input provided by the patient) can be detected. For instance, in a situation where the user 23 claims to feel nervous, he may be presented with some relaxing content, and afterwards the user claims to feel relaxed. However, the physiological measurements indicate a state of higher anxiety compared to what is normal for that user, i.e., baseline data.

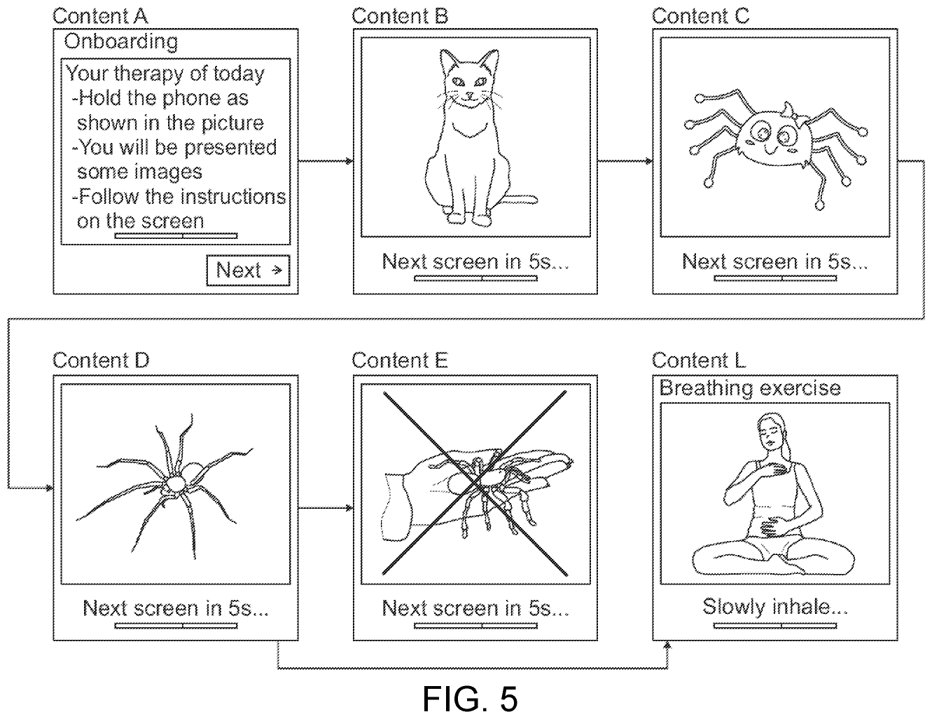

FIG. 5 shows an exemplary series of contents to be presented to a user suffering from a fear of spiders based on the determined reaction of the user.

First, content A is presented to the user 23 on his smartphone 10. Content A has emotionally neutral content relating to instructions for the interaction with the smartphone. When viewing content A, the user is determined to be in a relaxed state and shows no significant physiological reaction.

Second, content B is presented to the user 23 on his smartphone 10. Content B is a picture of a cat and thus is expected to be emotionally neutral or pleasant to the user suffering from fear of spiders. When viewing content B, the user is determined also to be in a relaxed state and shows no significant physiological reaction.

Third, content C is presented to the user 23 on his smartphone 10. Content C is a cartoon picture of a spider and thus is expected to elicit only a very mild reaction in the user suffering from a fear of spiders. When viewing content C, the system determines that the user 23 is in a state of very mild anxiety and shows only a very mild physiological reaction, e.g., a small increase in heart rate. The reaction of the user is still within a defined tolerance range, so the next content in the series is displayed.

Thus, content D is presented to the user 23 on his smartphone 10. Content D is a realistic picture of a spider and thus is expected to elicit a moderate reaction in the user suffering from a fear of spiders. When viewing content D, the system determines that the user is in a state of strong anxiety and shows a strong significant physiological reaction, e.g., a large increase in heart rate. The reaction of the user to content D exceeds the defined tolerance range. As the reaction of the user to content D exceeds the defined tolerance range, the next content in the defined series is not presented to the user, but rather a different content is displayed, in this case content L is selected to be displayed next. The reason for adapting the displayed content is because the content in the series is arranged to be increasingly provocative for an exposure therapy of a user suffering from a fear of spiders. However, if a certain level of exposure has been achieved and thus a certain reaction of the user outside of the tolerance range has been achieved, then it is not desirable to induce more fear in the user, and the content to be displayed is adapted accordingly.

After content D is displayed, thus content L is presented to the user instead of the originally planned content E. In this case, content L is a program guiding the user through a breathing exercise to calm the user. Content E which originally had been planned to be presented after content D is not presented because this image of a spider sitting on a hand is expected to be even more provocative to the user, and the user's reaction to content D already exceeded the tolerance range.

Although the present invention has been described in connection with certain specific embodiments for instructional purposes, the present invention is not limited thereto. Accordingly, various modifications, adaptations, and combinations of various features of the described embodiments can be practiced without departing from the scope of the invention as set forth in the claims.

1-11. (canceled)

12. A method for delivering a mental health treatment to a user adjusting content presented to the user based on a physiological parameter of the user, the method comprising:
presenting a first content to the user using an output unit;
monitoring the physiological parameter of the user during a time period during which the first content is presented to the user using the output unit in order to obtain monitoring data from the user;
synchronizing the monitoring data regarding the physiological parameter unit during the time period during which the first content is presented by the output unit via a synchronization unit to link in time the monitoring data to the first content, wherein the monitoring data is linked to the first content using timestamps;
analyzing the monitoring data and linking the monitoring data to the first content to determine a real-time reaction of the user immediately upon initially being presented the first content; and
controlling the output unit to present a second content to the user, wherein the second content is selected based on the reaction of the user to the first content in order to achieve a desired change in the physiological parameter that corresponds to a lower anxiety of the user.

13. (canceled)

14. The method of claim 12, wherein the physiological parameter is selected from the group consisting of: heart rate, respiration rate, pupil dilation, body temperature, and skin conductivity.

15. (canceled)

16. The method of claim 12, further comprising:
detecting changes in the physiological parameter relative to the physiological parameter monitored at an earlier time; and
determining the reaction of the user to the first content based on the detected changes in the physiological parameter.

17. (canceled)

18. The method of claim 12, wherein the second content is selected to elicit a desired reaction of the user.

19. The method of claim 18, wherein the second content is selected so as to elicit a physiological reaction of the user that is stronger than that elicited by the first content if the physiological reaction of the user to the first content falls within a predetermined tolerance range, and wherein the second content is selected so as to elicit a physiological reaction of the user that is milder than that elicited by the first content if the physiological reaction of the user to the first content falls outside the predetermined tolerance range.

20. The method of claim 12, wherein the synchronization unit is part of a device that includes a clock that generates a clock signal, and wherein the clock signal is used to synchronize the monitoring data with the first content presented to the user by the output unit.

21. A method for delivering a mental health treatment to a patient by generating customized content based on a physiological parameter of the patient, the method comprising:
presenting a first content to the patient;
measuring the physiological parameter of the patient at a time instant at which the first content is presented to the patient in order to obtain monitoring data from the patient;
synchronizing the monitoring data at the time instant at which the first content was presented so as to link in time the monitoring data to the first content, wherein the monitoring data is linked to the first content using timestamps;
analyzing the monitoring data to determine a real-time reaction of the patient immediately upon initially being presented the first content; and
presenting a second content to the patient, wherein the second content is selected based on the real-time reaction of the patient to the first content so as to achieve a desired change in the measured physiological parameter of the patient.

22. The method of claim 21, wherein the first content to which the real-time reaction of the patient is determined is a single image.

23. The method of claim 21, wherein the physiological parameter that is used to obtain the monitoring data is measured within one second of the first content first being presented to the patient.

24. The method of claim 21, wherein the real-time reaction of the patient to the first content is an increase in an instantaneous heart rate of the patient.

25. The method of claim 21, wherein the mental health treatment is an exposure therapy, and wherein the desired change in the measured physiological parameter of the patient corresponds to reducing an increase in anxiety exhibited when the patient is exposed to a predetermined stimulus.

26. The method of claim 21, wherein the desired change in the measured physiological parameter of the patient corresponds to a decrease in agitation of the patient.

27. The method of claim 21, wherein the physiological parameter is selected from the group consisting of: instantaneous heart rate, average heart rate, heart rate variability, respiration rate, pupil dilation, body temperature, and skin conductivity.

28. The method of claim 21, wherein the physiological parameter is a heart rate of the patient, wherein the monitoring data is a heart rate value of the patient at the time instant, wherein the first content is a single image displayed to the patient at the time instant, and wherein the heart rate value at the time instant is synchronized to the single image that was presented at the time instant.

29. The method of claim 28, wherein the second content is selected based on the real-time reaction of the heart rate of the patient to the single image presented to the patient at the time instant.

30. The method of claim 28, wherein the heart rate value at the time instant is synchronized to the single image that was presented at the time instant using a common timestamp.

31. The method of claim 28, wherein the desired change in the measured physiological parameter of the patient is a decrease in a heart rate of the patient.

32. The method of claim 21, further comprising:
detecting changes in the physiological parameter relative to the physiological parameter monitored at an earlier time; and
determining the real-time reaction of the patient to the first content based on the changes detected in the physiological parameter.

33. The method of claim 21, wherein a clock signal is used to synchronize the monitoring data with the first content that was presented to the patient at the time instant at which the physiological parameter of the patient was measured.

34. A method for delivering a mental health treatment via a smartphone to a patient, the method comprising:
presenting a first content to the patient;
measuring a physiological parameter of the patient at a time instant at which the first content is first presented to the patient so as to calculate a physiological parameter value;
synchronizing the first content to the physiological parameter value corresponding to the time instant at which the first content was first presented, wherein the synchronizing is performed using timestamps;
analyzing the physiological parameter value to determine a reaction of the patient to the first content that occurs immediately as the first content is first presented to the patient; and
presenting a second content to the patient after the reaction of the patient to the first content is determined, wherein the second content is selected based on the reaction of the patient to the first content so as to achieve a desired change in the measured physiological parameter of the patient.

35. The method of claim 34, wherein the desired change in the measured physiological parameter of the patient corresponds to a decrease in anxiety of the patient.

36. The method of claim 34, wherein the second content is selected so as to elicit a stronger physiological reaction from the patient than that elicited by the first content if the physiological reaction of the patient to the first content falls within a predetermined tolerance range.

37. The method of claim 34, wherein the physiological parameter is an instantaneous heart rate of the patient at the time instant, wherein the instantaneous heart rate is measured using a camera of a smartphone directed at a forehead of the patient, and wherein the instantaneous heart rate is calculated based on a photoplethysmogram (PPG) signal extracted from a video stream depicting the forehead of the patient acquired up to the time instant.

38. The method of claim 34, wherein the physiological parameter that is used to calculate the physiological parameter value is measured within one second of the time instant at which the first content was first presented to the patient.
